(19) United States
(12) Patent Application Publication
(10) Pub. No.: US 2012/0265167 A1
(43) Pub. Date: Oct. 18, 2012

(54) BIOCOMPATIBLE MATERIAL FOR ORTHOPEDIC USES

(76) Inventors: Rush E. Simonson, Jupiter, FL (US); Peter M. Simonson, Longboat Key, FL (US)

(21) Appl. No.: 13/330,542

(22) Filed: Dec. 19, 2011

Related U.S. Application Data
(60) Provisional application No. 61/425,648, filed on Dec. 21, 2010.
(60) Provisional application No. 61/449,532, filed on Mar. 4, 2011.

(51) Int. Cl. A61F 2/28; A61M 25/10; A61B 17/58

(52) U.S. Cl. 604/506; 623/23.56; 606/94

(57) ABSTRACT

Biocompatible material for bone repair, especially vertebral bone repair, preferably has three components. The first component is silicon nitride ceramic spheres or shells that can be polyhedral in shape. When grouped together, these ceramic spheres or shells form tessellates having a similar degree of stiffness, strain and stress resistance to cancellous bone. The second component comprises various bioactive factors that are preferably osteoconductive, osteoinductive and osteogenic. The third component is a liquid or gel that combines with the first and second components to form a composite.

### CROSS-REFERENCE TO RELATED APPLICATIONS

This application claims the benefit of priority to U.S. Provisional Application No. 61/425,648, filed on Dec. 21, 2010, and U.S. Provisional Application No. 61,449,532, filed on Mar. 4, 2011, the disclosures of which are incorporated herein by reference in their entirety for all purposes.

### FIELD OF INVENTION

The invention relates to a biocompatible material that promotes new bone differentiation, growth and fusion. More specifically, the present invention relates to composition and methods for repairing, reinforcing and treating osteoporotic, compressed or fractured bone. The invention also provides a system for repairing or replacing intervertebral discs with the biocompatible material to restore intervertebral disc space and promote fusion.

### BACKGROUND OF THE INVENTION

Osteoporosis, afflicting 55% of Americans aged 50 and above, is a major cause of vertebra fractures. Of these patients, approximately 80% are women and, if over 50, between 35-50% of these women have at least one fractured vertebra. In the United States, 700,000 vertebral fractures from osteoporosis occur annually often leading to kyphosis—a pathological curving of the spine caused by a spinal deformity where a number of spinal vertebrae lose some or all of their natural lordotic profile. Kyphosis is not only the result of degenerative diseases such as arthritis or osteoporosis but also developmental problems, compression fractures and/or trauma. Approximately one third of these patients develop chronic, debilitating pain that does not respond well to the conservative treatment of rest.

The current medical options for alleviating pain due to vertebral fracture include vertebroplasty and kyphoplasty—minimally invasive surgical techniques where balloons are inserted into the vertebral body to expand and compress bone tissue by creating a cavity within the vertebra. Using percutaneous techniques, bone cement is injected into the cavity. Ideally, this bone cement restores the mechanical integrity of the vertebral body by stabilizing the cortical bone fracture, thereby relieving pain.

There are generally two different approaches to vertebroplasty and kyphoplasty—transpedicular and posterolateral. If a transpedicular approach is taken, a catheter 6 shown in FIG. 1 is inserted into the vertebral body 2 by drilling an access portal through either pedicle 4. The catheter 6, shown with an un-inflated balloon 8 attached around its distal end, penetrates either one of the left or right pedicles 4 and reaches the vertebral body. When expanded, the balloon 8 assumes a cylindrical shape around the catheter 6. In most cases, the transpedicular approach is desirable because the pedicle comprises about 5 to 20 millimeters of cortical bone surrounding a small center of cancellous bone thereby making an excellent access portal.

The posterolateral approach uses a catheter that is inserted directly into the vertebral body by drilling an access portal directly into the cortical bone. As shown in FIG. 2, a catheter 10 contains an un-inflated balloon 12 around its distal end. When expanded, the balloon 12 expands outward from the distal end of the catheter 10. A posterolateral approach is less desirable because the cortical bone is thinner and may have already experienced compression. Furthermore, a posterolateral procedure involves a costotransversectomy where an incision is made along the paraspinous muscles, spanning about four or five ribs. The rib and transverse process are then re-sected at one to four levels followed by careful retraction of the pleura that expose the vertebral bodies and pedicles.

In the majority of cases, both procedures are effective in relieving pain by preventing micro-movement of the cancellous bone inside the vertebrae. They do so by providing mechanical stabilization of existing micro-fractures within the cortical bone. To illustrate this point, FIG. 3 A-E shows a prior art schematic of a transpedicular kyphoplasty procedure using a commercial product similar to the Kyphon® Balloon Kyphoplasty sold by Medtronic and described in U.S. Pat. Nos. 4,969,888 and 5,108,404 by Scholten et al. FIG. 3A is a side view of a vertebral body showing the initial insertion of an elliptical balloon into the damaged vertebral body before the balloon is inflated. FIGS. 3B and 3C shows the gradual inflation of the balloon 14 to form a cavity 16 in the cancellous bone of the vertebral body. FIG. 3C also shows the initial stage where bone cement is injected into the cavity 16. Finally, FIG. 3E shows the cavity 18 after bone cement has hardened.

The most common bone cement is polymethmethacrylate or PMMA. PMMA is a polymeric material that the surgeon mixes during the surgical procedure and injects into the vertebral body. Most commercial PMMA bone cements are available in two separate components: a powder comprised principally of pre-polymer balls of polymethmethacrylate (PMMA) and a liquid of the monomer, generally methyl methylmethacrylate (MMA), reacting in the presence of a polymerization activator. For in vivo use, a reaction initiator is added to avoid high reactive temperatures since the polymerization reaction is exothermic. An initiator such as benzoyl peroxide is generally incorporated with the powder while the liquid contains a chemical activator (catalyst) usually dimethylparatoluidine. The polymerization reaction begins when the two components are mixed. In order to avoid spontaneous polymerization, a stabilizer such as hydroquinone is used. In order to display the bone cement, a radioopaque substance such as barium sulfate or zirconium dioxide is added. For the most part, these binary compositions of bone cements were originally designed for the attachment of implants and sealing of prostheses. When using such bone cements in percutaneous surgery, they present certain risks and problems associated with the toxicity of methylmethacrylate. This is especially true when such cement is applied with pressure to make it flow through a catheter since it has to maintain this fluidity long enough to give the surgeon time to operate. Furthermore, the exothermic polymerization process often leads to substantial damage of the surrounding tissue. Handling is also a problem because the final preparation of the PMMA mixture is performed in situ where individual components are measured, mixed to a homogenous mixture and filled into the appropriate device for application, which, in the case of vertebroplasty, is usually a syringe. In general, PMMA is far from the ideal material for bone augmentation and, in particular, for application in vertebroplasty.

The most dangerous risk and problem in using PMMA is the extraosseous leakage of bone cement reported in 70% of these procedures. As shown in FIG. 3C, this leakage 20 is due to the fact that bone cement is injected under pressure into a closed space inside fractured bone. If already fractured or collapsed, such compaction applies substantial pressure (from 50 to 300 psi) to the inner cancellous bone, which has the effect of furthering damaging perfectly good and healthy outer cortical bone. If there is initial leakage 20 (FIG. 3C) into either the anterior or posterior columns of the vertebral body, the highly toxic methylmethacrylate may leach out into the blood stream causing blood pressure drop and migration into the veins. If the anterior longitudinal ligament 24 does not stop major leakage 22 shown in FIG. 3D, this extravasation of bone cement can have serious ramifications. While not frequently observed, pulmonary embolism leading to cardiac failure has been reported.

Even after successful injection and polymerization, PMMA can cause further complications. When hardened, PMMA is very hard and causes increased rigidity of the vertebral body. In comparison to cancellous bone tissue (0.5 GPa), the rigid modulus of PMMA (1-3 GPa) can lead to stiffness, strain and stress compression inconsistencies in 26% of kyphoplasty cases. Such modulus differences can cause stress, fracture and/or collapse of the superior (top) or inferior (bottom) vertebra and are especially egregious when considering compressive strength of a healthy vertebra as compared to an osteoporotic or damaged vertebra. Under continuous loading, it has also been reported that PMMA cracks and, when it does so, it seeps chemicals that become toxic to both new bone formation and, of course, the patient's general health. Interestingly, PMMA and other polymers have also found to harbor infectious agents.

Similar polymeric materials are also used in repairing or replacing intervertebral discs. As shown in FIG, 13A, intervertebral discs 63 are located between adjacent vertebrae in the spine and provide structural support for the spine as well as distribute forces exerted on the spinal column. Such discs contain a stiffer outer portion (annulus fibrosus) that provides peripheral mechanical support and torsional resistance. An inner portion (nucleus pulpous) contains a softer nuclear material to resist hydrostatic pressure. Most intervertebral discs, however, are susceptible to a number of injuries. With age and constant pressure, disc herniation 68 is common. Herniation starts when the nucleus begins to extrude 70 through an opening often where the herniated disc impinges on nerve roots in the spine. In most cases, the posterior and posterolateral portions of the discs are most susceptible to such herniation.

Current treatments for intervertebral disc injury include nuclear prostheses or disc spacers. There are, in fact, numerous varieties of prosthetic nuclear implants in the art. For example, there is the total disc replacement by Sulzer. Its BAK® Interbody Fusion System uses hollow, threaded cylinders that are implanted between the vertebrae. These implants are packed with bone graft to facilitate the growth and fusion of vertebral bone. Other intervertebral prosthetic implants can be formed from flowable polyurethane compositions that are delivered into the intervertebral spaces where it reacts in situ to form solid polyurethane (PU) and are fully cured under normal physiological conditions. In some cases, these polymeric compositions are delivered through inflatable balloons or molds where they create an interior cavity to receive the curable composition. Similar to PMMA, polyurethane (PU) is formed from toxic compounds such as diisocyanates including toluene diisocyanates, napthylene diisocyanates, phenylene diisocyanates, xylene diisocyantes, diphenylmethane diisocyanates and other aromatic and aliphatic polyisocyanates. Like PMMA, any extravasation of PU may have serious medical ramifications.

Since PMMA and PU are not optimal cements or fillers, numerous groups have examined more bioactive cements, either calcium phosphate cements or polymeric cements containing bioactive ceramics for both vertebral and intervertebral fusions. While the bioactivity of these materials is an improvement over PMMA and PU, the mechanical properties of these cements have been questioned for sufficient compressive strength and high modulus mismatches to cancellous bone or intervertebral discs. Recently, injectible bone substitutes combining polymers and bioactive ceramics have been described. One case, for example, incorporated various bioactive glass beads and calcium phosphate granules to reinforce the polymer, but the cement came apart from the beads. In another proposal, hydrogels were suggested but their permanence was questionable.

In summary, there is a need for a truly biocompatible material that doesn't seep toxic chemicals and, instead, promotes healthy bone differentiation and growth. A characteristic of a new biocompatible material should be that it does not fail from cyclic loading and, of course, does not harbor infectious agents. An ideal material might also augment the natural mechanical properties of bone while promoting healthy differentiation and growth of osteoporotic, compressed or fractured vertebral bodies or discs, especially with the growing worldwide elderly population.

### BRIEF SUMMARY OF THE INVENTION

The present invention provides biocompatible materials for percutaneous surgical use and, in particular, for filling and cementing bone cavities and intervertebral disc spaces. The biocompatible materials of the present invention possess fluidity, fluoroscopic opacity and, in one embodiment, has stress resistance similar to cancellous bone and intervertebral discs. It also comprises bioactive adjuvants or factors that promote vertebrate bone differentiation, growth and fusion.

In a preferred form, a first component of this biocompatible material is silicon nitride doped with other oxides, such as yttrium oxide and/or alumina. Under high temperature and pressure, a silicon nitride ceramic sphere is made. Such a ceramic sphere possesses a high load bearing capability, strong bio-mimetic scaffolding, and excellent radio-opaque characteristics. Furthermore, the porosity and pore size of this ceramic sphere allows for optimal bone ingress, high vacularization and mechanical properties similar to cancellous bone. The shapes of such ceramics spheres are preferably hexagonal, octahededronal or any other polyhedral combination. When grouped or stacked together, these ceramics spheres form tessellates that, in combination with other components, provide a similar degree of stiffness, strain and stress resistance to cancellous bone. These polyhedral shapes also allow the ceramic spheres to roll and tumble like beads or balls especially during delivery through a catheter tube during vertebroplasy, kyphonplasty and discectomy.

In another preferred embodiment, a second component can be added to the first component comprising a plurality of various bioactive inorganic growth factors that are osteoconductive, osteoinductive and osteogenic. Such inorganic compounds may include known osteoconductive compounds, such as calcium phosphate, hydroxy-apatite or tri-calcium phosphate. Demineralized or lyophilized segments of bone (demineralized bone) also induce new bone formation. Preferred osteoinductive and osteogenic biomaterials may further include natural or synthetic therapeutic agents, such as bone morphorgenic proteins (BMPs), growth factors, bone marrow aspirate, stem cells, progenitor cells. Additionally, amniotic fluid, antibiotics or any other bone growth enhancing materials or beneficial therapeutic agents may be used.

The third component that can be added to the first and second components is a plurality of liquid or gel fillers such as collagen, glycoaminoglycans, and hyrodgels that mix, combine and lubricate the previous components into a composite. The third component gives the composite viscosity thereby easing the delivery of such ex-vivo biocompatible materials through a catheter to the cancellous core or intervertebral disc space.

In a preferred embodiment, a silicon nitride shell containing all three components surrounds a silicone center thereby making an elastic ceramic sphere possessing the compressive strength and Young's modulus E similar to cancellous bone or intervertebral discs.

The present invention has numerous uses. In its preferred use, the components of this biocompatible material may fill, augment, repair or replace damaged vertebrae and/or intervertebral disc spaces. The biocompatibility of the present invention is an improvement over PMMA and PU because the risks and problems associated with the toxicity of methylmethacrylate or polyisocyanates are mitigated. The present invention may also be used for repairing or replacing intervertebral discs with either the biocompatible material and balloon prosthesis or both to restore intervertebral disc space height. In another use, this biocompatible material may help repair, reinforce and/or treat other types of fractured and/or diseased bone including filling defects, cavities and gaps of fractured or diseased long bones. In another preferred embodiment, the biocompatible material can be stringed together or arranged in a matrix mesh to promote differentiation and growth of bone during bone fusion, especially in posterolateral spinal bone fusion.

### BRIEF DESCRIPTION OF THE DRAWINGS

FIG. 1 is a top view of a lumbar vertebra with a prior art balloon catheter deployed by the transpedicular process prior to inflation.

FIG. 2 is a top view of the lumbar vertebra with a prior art balloon catheter deployed by posterolateral process prior to inflation.

FIG. 3A is a prior art schematic side view of a vertebral body showing the initial insertion of an elliptical balloon catheter into the vertebral body before inflation of the balloon.

FIG. 3B is a similar view to FIG. 3A but shows inflation of the balloon to form a cavity in the cancellous bone of the vertebral body.

FIG. 3C is a view similar to FIG. 3B but shows the balloon removed and the injection of methyl methacrylate cement into the newly created cavity.

FIG. 3D is a similar view to FIG. 3C but shows an exploded view of extraosseous cement leakage and extravasation of bone cement into the body.

FIG. 4A shows various polyhedral spheres.

FIG. 4B shows the tessellation of polyhedral ceramic spheres.

FIG. 5A shows surface pores and the porosity of the ceramic sphere.

FIG. 5B shows an exploded view of the porous surface of the ceramic sphere coated with osteoinductive biomaterials.

FIG. 5C shows an exploded view of the porosity of the ceramic sphere embedded with osteoconductive and osteogenic biomaterials.

FIG. 6 shows a spherical or hexagonal silicon nitride shell filled with silicone.

FIG. 7 is a side and cut-away view of a cavity inside a fractured and compressed vertebral body being filled with the biocompatible material of the present invention.

FIG. 8A is a side, cut-away and exploded view of a ceramic sphere tessellate inside a vertebral body.

FIG. 8B shows a coated ceramic sphere pore with new cortical bone on its surface.

FIG. 8C is an exploded view of ceramic sphere ingress with new cancellous bone.

FIG. 9A shows random distribution of ceramic spheres in a bone fusion bed.

FIG. 9B is a string of ceramic spheres in a bone fusion bed.

FIG. 9C is a mesh of ceramic spheres in a fusion bed.

FIG. 10 is a string and mesh of ceramic spheres on either side of a posterolateral vertebral fusion bed.

FIG. 11 shows a flexible rod with silicon nitride ceramic blocks.

FIG. 12 shows flexible rods with silicon nitride ceramic blocks or spheres embedded in a bone graft fusion.

FIG. 13A shows a number of herniated intervertebral discs.

FIG. 13B shows a conventional intervertebral disc space distractor and a balloon distractor approach from the posterior spine.

FIG. 13C shows a rotate cutter performing bilateral hemilaminectomy and discectomy.

FIG. 13D shows the insertion of a solid disc implant and biocompatible material into the intervertebral disc space.

FIG. 14 shows a discectomy, biocompatible material insertion and a balloon prosthesis approach from the lateral spine.

FIG. 15A shows an intervertebral balloon prosthesis being filled with biocompatible material.

FIG. 15B shows an intervertebral balloon prosthesis being absorbed during fusion.

FIG. 16 shows a balloon prosthesis restoring intervertebral disc space and promoting fusion with a supplemental segmental internal fixation device.

FIG. 17 shows a femur being filled with the biocompatible material.

FIG. 18 A-C shows various embodiments of an instrument that fills defects, cavities and/or gaps with biocompatible material.

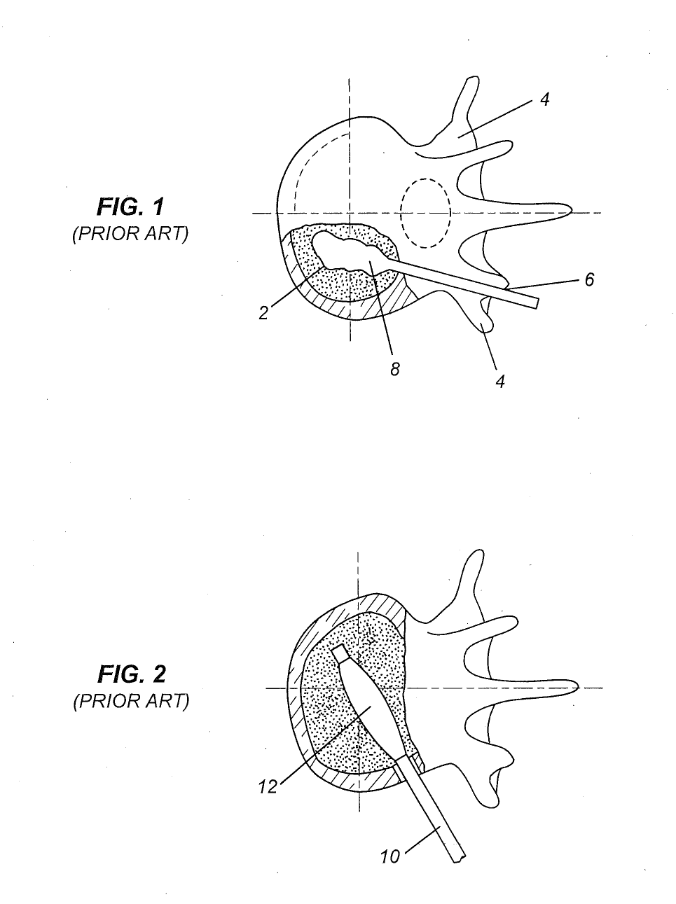

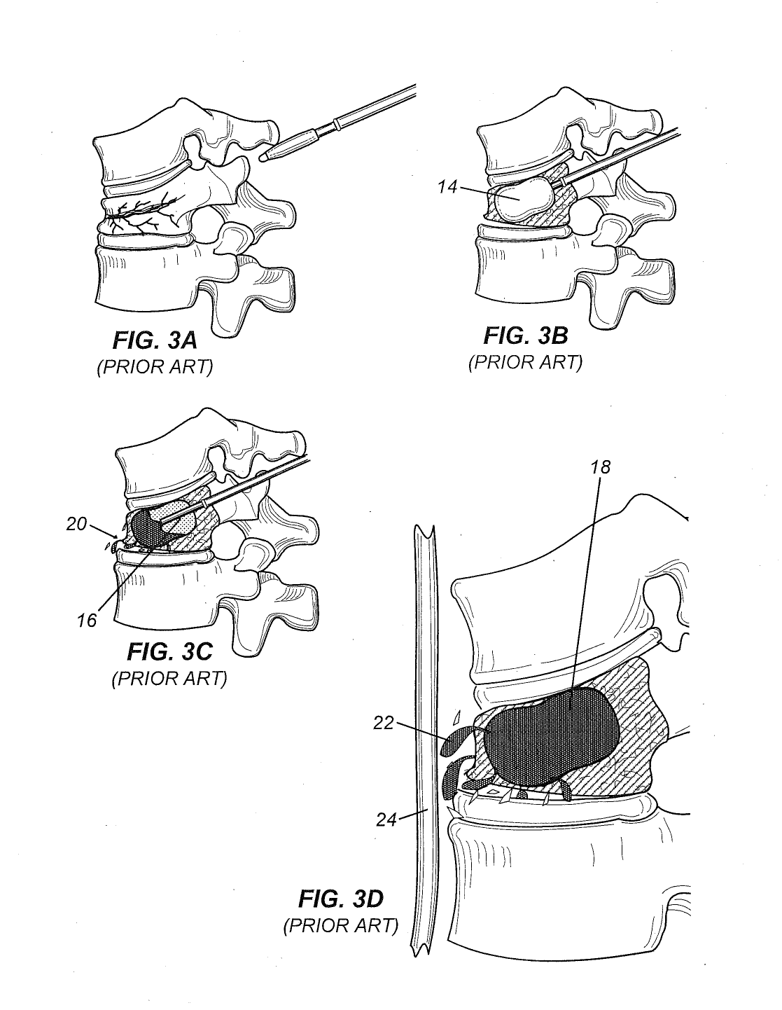

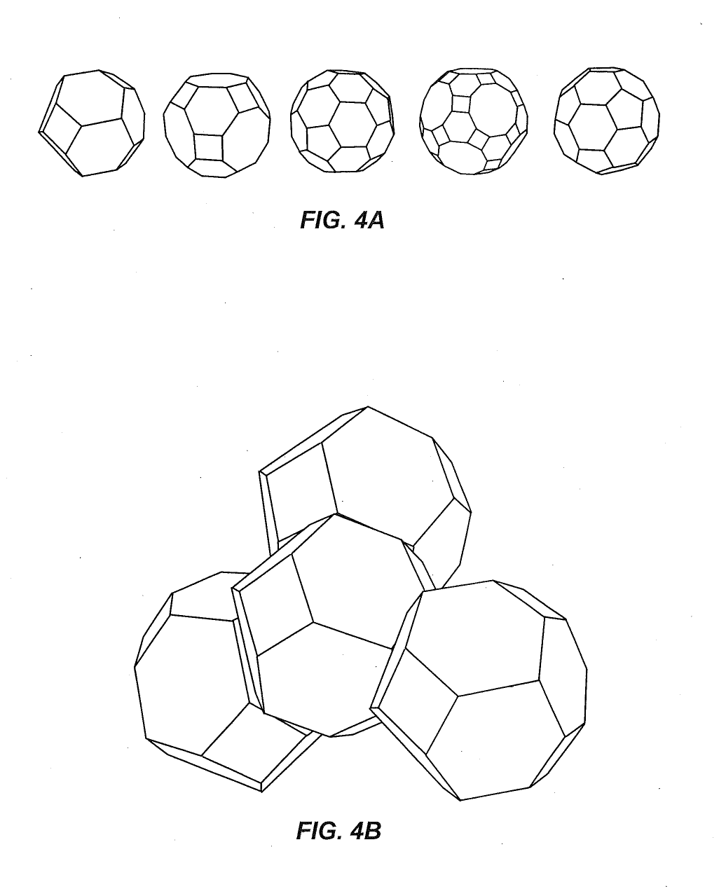

### DETAILED DESCRIPTION OF THE INVENTION

I. Introduction

The present invention comprises one or more biocompatible materials for use in orthopedics. It is designed to reduce the pain associated with fractured bone or ruptured intervertebral discs and improve the mechanical properties of osteoporotic, compressed or fractured bone and intervertebral discs. More importantly, the present invention promotes osteoblastic activity and vascular penetration for new bone differentiation, growth and fusion. The present invention may substitute for PMMA or PU and eliminate the adverse effects of such existing bone cements or fillers. Instead of being toxic, the present invention is biocompatible and possesses the ability to elicit the appropriate biological host response. Contrary to other cements, the present invention interfaces with biological systems to treat, grow, repair and/or replace osteoporotic, compressed or fractured bone and intervertebral discs. The biocompatible material of the present invention comprises a number of bio-mimetic and bioactive components to improve and strengthen mechanical stabilization as well as promoting bone differentiation, growth and fusion. The preferred biomaterial includes at least three components. The first component is a number of ceramic spheres preferably made from silicon nitrate, its analogs and/or derivatives. When combined, these ceramic spheres tessellate together with their polyhedral sides interfacing with one another. Together, these ceramic spheres possess load bearing, compressive and mechanical properties superior to PMMA and other polymers. Furthermore, the ceramic spheres tessellate to provide favorable bio-mimetic scaffolding for in-growth and rapid integration with host bone. In particular, the surface porosity and pore size of the spherical shaped ceramic surface allows for optimal ingress of bone growth and vascularization. Such in-growth and vascularization can be further augmented by the addition of a second component. The preferable second component consists of various bioactive materials including inorganic compounds and biological growth factors. These second components are preferably osteoconductive, osteoinductive and osteogenic compounds that can easily coat or reside in the pores and ingresses of the ceramic sphere. The third component is a low viscous liquid or gel mixed with the first and second components. It also serves as a lubricant. The third component is preferably collagen, glycoaminoglycans, hyrodgels or other biological liquid or gel filler that can easily combine with the first and second components. Additionally, the third component gives the composition viscosity thereby easing the delivery of such ex-vivo biocompatible materials through a catheter to the cancellous core or intervertebral disc space during vertebroplasty, kyphonplasty or discectomy. The third component may further be a liquid or gel to form a composite from the injectable biomaterials. In combination, it is this biocompatible mixture of material that provides compressive strength and Young's modulus E similar to cancellous bone or the outer portion (catheter fibrosus) of intervertebral discs. In summary, the biocompatible material of the present invention will first mechanically stabilize the bone and intervertebral discs temporarily and, second, gives the osteoconductive and osteoinductive biomaterials time to take effect and promote new bone growth, differentiation and fusion in the longer term.

II. Definitions

“Augmentation” means the act of making larger and particularly stronger by the addition and increase of tissue.

“Bioactive” means a substance that beneficially interacts with or has a positive effect on tissue and cells.

“Biocompatible” refers to biomaterials that elicit an appropriate host response without any adverse effects.

“Biomaterials” refers to any material that supports, augments or grows biological tissue.

“Biomimetic” means the use of biological methods applied to engineering systems or materials.

“Ceramic” refers to an inorganic and non-metallic solid prepared by high temperature, pressure and subsequent cooling.

“Collagen” means a substance made of naturally occurring proteins and is the main component of bone.

“Composite” refers to a mixture of components with covalent, non-covalent and ionic bonds to form tessellates that imparts stiffness similar to cancellous bone.

“Compression Strength” means the maximum stress a material can sustain under crush loading.

“Differentiation” means the process by which immature cells, such as stem cells, becomes a specialized cell.

“Exothermic” means a chemical reaction that gives off heat to its surroundings.

“Extravasation” means the leakage of infused substances into the vasculature.

“Ex-vivo” means outside the body.

“Glycoaminoglycans” means long un-branched polysaccharides consisting of a repeating disaccharide unit.

“Hydrogel” refers to a class of polymeric material that swells in an aqueous medium but does not dissolve.

“In-situ” means exactly in the place where it occurs.

“Intervertebral” refers to the space between vertebrae.

“Intravertebral” refers to the space inside vertebrae.

“In-vitro” means an artificial environment outside the living organism.

“In-vivo” means inside a living organism.

“Modulus” means a measure of tensile stiffness of an elastic material.

“Morphogenesis” means the differentiation and growth of tissue to make structures in an organism.

“Osteoblastic” means the growth of a mononucleate cell from which bone develops.

“Osteoconductive” means a passive process by which bone grows on a surface.

“Osteoinductive” means an active biologic response to chemical signals to induce bone formation

“Osteogenic” means the formation and development of bone.

“Percutaneous” means taking place through the skin.

“Radioopaque” means impenetrable to X-rays and other radiation, thereby making it visible on radiographic images.

“Sphere” refers to a round geometrical object in three-dimensional space and, as used herein, may be non-symmetrical around its center (e.g., including polyhedral structures).

“Stiffness” is a measure of resistance of plastic to bending and is measured by the Young's modulus E.

“Strain” is a change per unit length in the linear direction.

“Stress” is defined as the load divided by the area through which it acts and is measured in units of a Pascal (GPa, MPa or Pa). One Pa is equal to 1 kg/ms²

“Tessellation or tessellate” refers a collection of polyhedral spheres that coalesce together with little gap or overlap between them.

“Vascular/Vascularization” means the formation of blood vessels and capillaries in living tissue.

“Young's modulus or modulus” is defined as the rate of change of strain as a function of stress and is measured in units of Pa or MPa. It is the slope of stress-strain and measures both the tensile modulus of elasticity and compressive modulus of elasticity.

III. Compositions

The composition of biomaterials for augmenting cortico-cancellous bone and replacing intervertebral discs contains at least one or two materials including ceramics, biologically active agents and/or additives, fillers, base or solvents. The biomaterials are mixed ex-vivo to form a composite preferably containing at least the first and second components to form the biocompatible material. The nature and structure of the components selected is based on the type of biocompatible material desired.

A. Ceramic

In a preferred embodiment, the first component consists of silicon nitride (Si₃N₄) formed by a direct reaction between silicon and nitrogen at high temperatures forming a hard ceramic having high strength, moderate thermal conductivity, low thermal expansion, high elastic modulus and usually high fracture strength. The first component also includes analogs and derivatives of silicon nitride compounds. A composition with these properties leads to excellent thermal shock resistance, ability to withstand high structure loads and superior wear resistance.

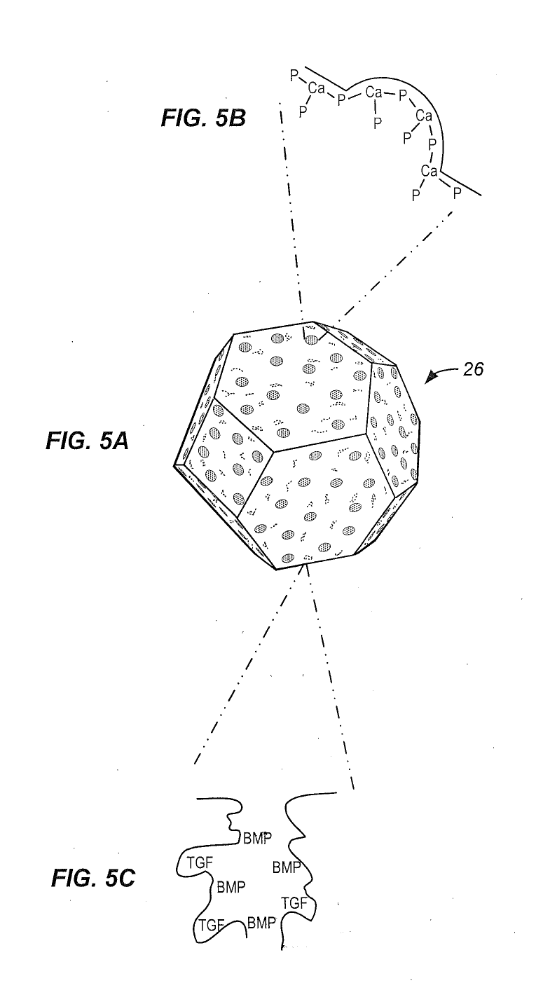

The preferred ceramic composition consists of powders of Si₃N₄ and may include dopants such as alumina, yttrium, magnesium oxide, and strontium oxide. The dopant amount is optimized to achieve certain density and mechanical properties. The homogenous powders are then preferably cold isostatic pressed at high Mega-Pascal (MPa) followed by sintering at a high temperature. A sintering temperature of approximately 1875° C. is preferred to achieve high density, absence of pores and a uniform fine-grained microstructure. To make the preferred bio-mimetic ceramic, lower temperatures can be used in sintering to produce a more porous ceramic. In a preferred form, the porosity of the ceramic may be 10% to 50% by volume with open pores distributed throughout and a pore size ranging from 5 to 500 microns. As shown in FIG. 5A, the porosity of the ceramic sphere 26 can be graduated from a relatively low porosity ceramic sphere emulating or mimicking the porosity of cortical bone to a higher porosity ceramic sphere emulating or mimicking the porosity of cancellous bone. These ceramics spheres possess both a high load bearing capability and strong bio-mimetic scaffolding necessary for in-growth and rapid integration with host bone. In a preferred embodiment, these ceramic spheres can be tailored for optimal ingress of vascularization, ease of carrying or delivering osteoconductive, osteoinductive and osteogenic factors to the cancellous core. The resultant porous ceramic spheres resemble the porous structure of either cortical or cancellous bone depending, for example, on the nature of the osteoporotic, compressive or fractured injury. Such cancellous structured ceramic is sold by, among others, Amedica, Inc. For example, Amedica's CSC™: Cancellous Structure Ceramic is a similar porous ceramic substrate from silicon nitride whose structure mimics that of natural cancellous bone.

To maximize compressive strength, the preferred shape of these ceramic spheres is spherical polyhedrals. Polyhedrals are a geometric solid in three dimensions with flat faces and straight edges. This may include polyhedral cubes and cylinders. As shown in FIG. 4A, the preferable polyhedrals are pentagonal, hexagonal, octahededronal or any other polyhedral shape combination that fit together without any gaps and are useful for constructing tessellates. As shown in FIG. 4B, a hexagon can form a regular tessellate having three hexagons around every vertex. When grouped or stacked together, these polyhedral spheres can form tessellates with their flat sides. As the polyhedral sides inter-lock, the frictional surfaces between ceramic spheres increase and compaction begins to occur. It is expected, however, that in vivo tessellation will not be perfect and have a number of gaps and overlaps. Their continued compaction in the vertebral body will, however, provide a degree of stiffness, strain and stress resistance. The gaps and overlaps can also increase the surface area to further promote the bio-mimetic scaffolding for in-growth and rapid integration with the host bone.

In the preferred embodiment, the size or diameter of these ceramic spheres are preferably in a range of about 0.5 millimeters (mm) to about 12 mm. Their size depends on what type of vertebrae they are to be deposited into. A useful measurement is the size of pedicle screws that are normally used during spinal fusion. Since cervical vertebrae pedicles average a width of 3-4 mm, the preferable diameter of the ceramic sphere may be between 1-2 mm. This diameter is below the usual 4 mm pedicle screw used for cervical vertebrae. Thoracic vertebrae pedicles are larger and average in width from 7-10 mm. In this case, the ceramic sphere may preferably be in a range of 3-4 mm. This diameter is below the usual 5-6 mm pedicle screw diameter used for thoracic vertebrae. For lumbar vertebrae, the ceramic sphere diameter may preferably be in the range of 5-7 mm since lumber pedicle width ranges from 10-16 mm. This sphere diameter is below the usual 7-8 mm pedicle screw used for lumbar vertebrae. Within this about 0.5 to 12 mm size range, these ceramic spheres can still roll or tumble through the insertion catheter. In order to do so, the catheter diameter may be similar to that of the specific pedicle screws used for those particular vertebrae. To facilitate their placement into the cancellous core of the vertebral body or intervertebral disc space, a lubricant and pressure may be used to move the ceramic spheres more easily through catheter. As for intervertebral disc space, sphere size may also differ based on the height of the disc space to be restored.

B. Biomaterials

A preferred second component comprises biomaterials selected for relatively high osteoinductive, osteoconductive and osteogenic properties to provide a rich and favorable environment to induce bone morphogenesis and differentiation. As shown in FIG. 5B, such inorganic biomaterials can easily coat the ceramic surface and shallow pores. Preferred surface coating materials comprise a re-absorbable material such as hydroxyapatite or a calcium phosphate (Ca—P). Optionally, hyroxy-apatite (HAP) or tri-calcium phosphate (TCP), which is similar to de-mineralized bone (a Ca deficient, carbonate containing apatite similar to Ca₁₀(PO₄)₆(OH₂)), can be used. The surface substrate and the porosity of the ceramic easily makes this bio-material surface coating possible by attaching to the relatively lower porosity regions residing and formed on the ceramic surface as shown in FIG. 5B.

In a further aspect of the invention, the biomaterials may additionally be comprised of one or more therapeutic agents to further enhance bone growth. Such bio-materials may include natural or synthetic therapeutic agents, such as bone morphorgenic proteins (BMPs), transforming growth factors (TGFs), bone marrow aspirate, stem cells and/or progenitor cells. Additionally, amniotic fluid, antibiotics or any other osteoconductive, osteoinductive, osteogenic, enhancing materials or therapeutic agents may be used. As mentioned, such bone growth factors may include the family of BMPs, including commercial BMPs such as BMP-2 sold by Medtronic and OP-1 BMP-7 sold by Stryker Biotech.

As shown in FIG. 5A-5C, ceramic spheres 26 may be advantageously coated or impregnated with one or more of these selected therapeutic agents. For example, autologous, synthetic or stem cell derived growth factors or proteins to further promote bone differentiation and growth may be used. The porosity of silicon nitride ceramics allow for the ingress of these bio-materials into these pore holes and, in particular, for growth factors to reside in the higher porosity regions shown in FIG. 5C. The ceramics spheres 26 can be tailored to allow for the ingress of morphogenesis and vascularization of the osteoporotic, compressed or fractured cortical or cancellous core. The preferred pore size for achieving bone in-growth ranged between 100 to 530 μm, with up to 55% porosity. Preferably, the resultant porous structure shown in FIG. 5A resembles the porous structure of both cortical and cancellous bone.

The combination of both of the two components—ceramics spheres and biomaterials—promotes vigorous bone formation at both the implant/host bone interface and within the pores and ingresses of the ceramic scaffold. This bone growth may be enhanced by the interconnection between the pores and the side interfaces of the polyhedral tessellate that forms within the cancellous core. At the host cortical bone/ceramic implant interface, new cortical bone can form at the surface. Furthermore, the pores, gaps and overlaps of the polyhedral surfaces and sides allow the spongy cancellous bone to penetrate deeper into the implant to promote vascular development. Silicon nitride ceramic scaffolding not only promotes primary and secondary bone growth but woven bone as well.

C. Filler

The third component of the present invention promotes mixing of the first two components to create a single-phase system. The third component may be a low viscous liquid to mix with the first and second components. Preferably, the third component is collagen, glycoaminoglycans, hyrodgels or other biological liquid or gel filler that can easily combine the first and second components. To provide the proper viscosity, sterile saline water may be used or added. In so doing, a low viscosity composition eases the delivery of such ex-vivo biocompatible material through a catheter to the cancellous core during, for example, vertebroplasty or kyphonplasty. In short, it serves as a lubricant. The third component may also be a gel to form a higher viscous composite material for the first and second components, thereby giving it more compressive strength. The third component preferably starts as a liquid to serve its lubrication function and may solidify into a gel-like composition to hold the composite together. Collagen, for example, may serve well because it can be easily denatured under low heat to form a liquid and reformed into a gelatin-like composition upon cooling (e.g., body temperature). With collagen as filler, the biomaterial composite may be suspended in a syringe or a more sophisticated injection device as a gel. A gel-like composition, for example, also promotes storage until needed for surgery. Upon warming, the collagen gel inside the syringe liquefies and can be easily plunged into the catheter. As a liquid, it serves as lubricant assisting the ceramic spheres through the catheter and into the inner cancellous core. Upon reaching its cancellous core designation, the collagen composite slowly cools and gels to hold and solidify the biocompatible material in the vertebral body. Hydrogels and glucoaminoglycans may also work as well.

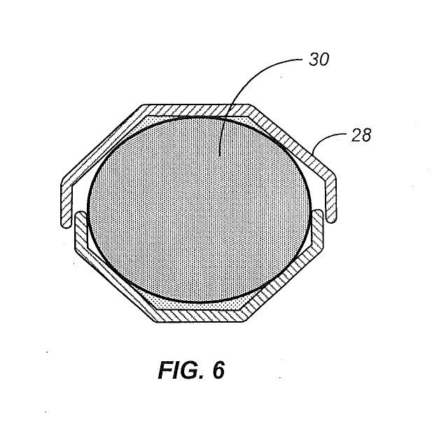

To make the biocompatible material have a modulus similar to cancellous bone or intervertebral discs, another preferred embodiment is shown in FIG. 6. In this embodiment, a silicon nitride ceramic shell 28, similar in shape and composition to the ceramic sphere 26 (FIG. 5A), surrounds a silicone filling 30 bonded to the shell. The ceramic shell 28 possesses elastic properties similar to cancellous bone. The ultimate compressive strength of the biocompatible ceramic shell 28 is preferably in the range between 10 MPa and 40 MPa and, even more preferably, between 15 MPa to 25 MPa. As with the ceramic sphere 26 (FIG. 5A), the bio-compatible c ceramic shell 28 may also be injected into defects, cavities and gaps to not only mechanically stabilize the bone and intervertebral disc space but, more importantly, to augment and grow new cancellous and cortical bone with its osteoconductive, osteoinductive and osteogenic properties. A surgeon can therefore choose between a bio-compatible material that has either a low or high modulus depending upon which bone defect, cavity, gap or disc space to be filled.

D. Uses

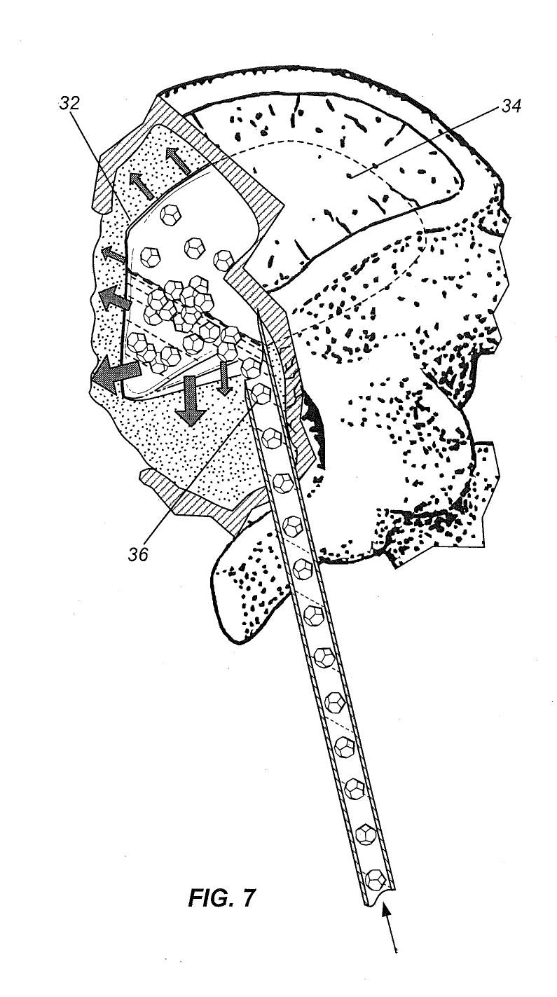

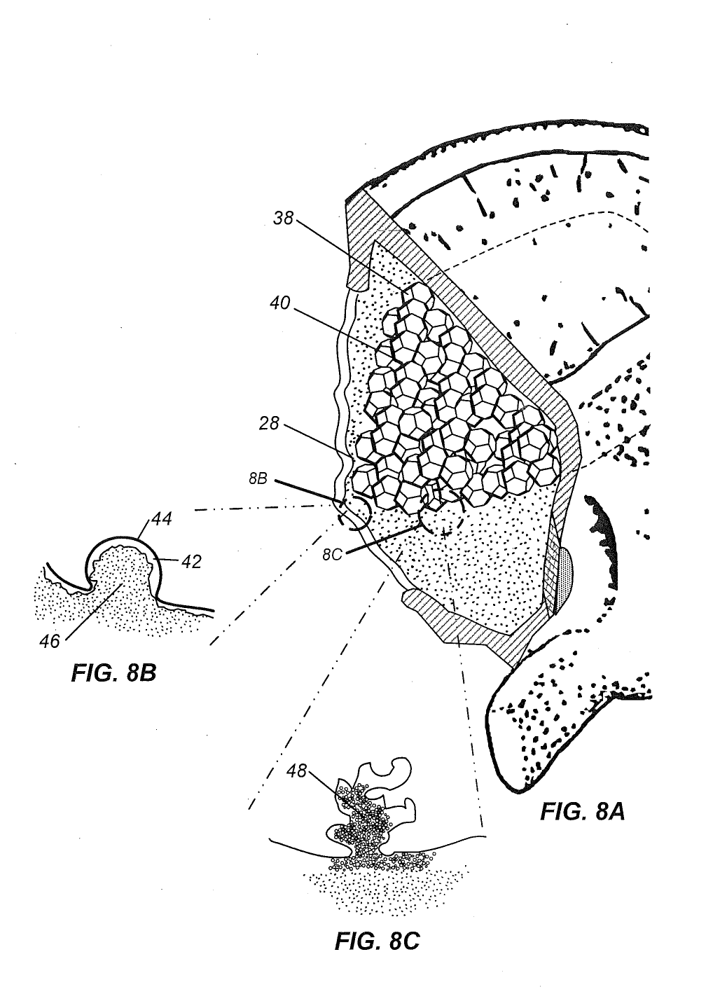

As previously described first, second and third components are preferably combined to make the biocompatible material, which has numerous uses. FIG. 7 shows, for example, a cavity 32 formed by a balloon during vertebralplasty in the cancellous bone of the vertebral body 34. In particular, FIG. 7 shows the initial stage where the biocompatible material 36 is injected into the cavity 32. The preferred embodiment in FIG. 8A shows how the ceramic spheres or shells 38 and its tessellate 40 restores intra-vertebral height and also provides the mechanical stability necessary to support osteoporotic, compressed and/or fractured vertebrae. The exploded view of FIG. 8B shows how the biomaterial coating 42 in the pore 44 of the ceramic sphere or shell promotes cortical bone growth and differentiation 46 near the periphery of the damaged intravertebral body or intervertebral disc. Furthermore, the exploded view of FIG. 8C shows how the embedded therapeutic biomaterials (FIG. 5C) within the ingresses of the ceramic sphere or shell promote cancellous bone growth and differentiation 48. In using the biocompatible material for vertebralplasty, a surgeon now has a choice between either the ceramic sphere or shell 38 embodiments based on the elasticity required.

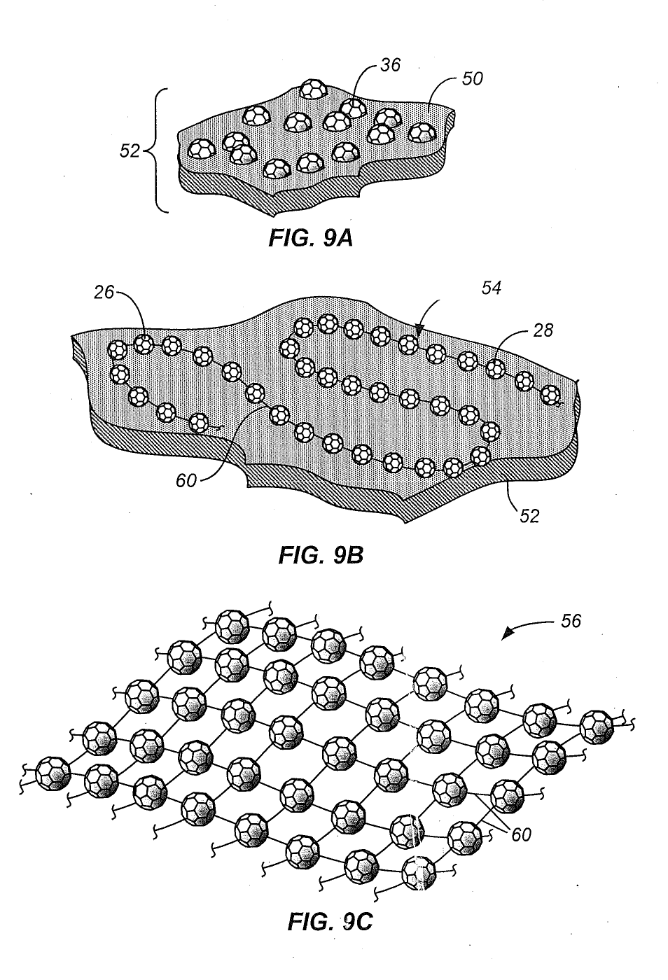

In another use, FIG. 9A shows how the biocompatible material can be used for inter-vertebral use especially during posterolateral fusion. Posterolateral fusion places bone graft between the transverse processes in the back of the spine. The biocompatible material 36 can be randomly placed and mixed throughout the posterolateral bone graft 50 to promote fusion. Since the fusion process typically takes 6-12 months after surgery, the biocompatible material 36 may promote fusion differentiation and growth thereby speeding up the fusion process. When the posterolateral fusion bed 52 is laid, the vertebrae are then often fixed with bone screws through the pedicles of each vertebra and connected to a spinal rod.

To avoid extraosseous leakage of the biocompatible material in any of the uses described herein, another preferred embodiment is shown in FIG. 9B. In this embodiment, the ceramic spheres 26 or ceramic shells 28 of the biocompatible material are threaded together making a flexible string 54 of the biocompatible material. This string embodiment can be furthered embodied by making a mesh 56 of the biocompatible material shown in FIG. 9C. To prevent extraosseous leakage, the biocompatible string 54 can be also used for filling the intravertebral bodies or intervertebral disc spaces.

The thread 60 of the bio-compatible string 54 or mesh 56 may be preferably made from absorbable or non-absorbables suture material selected from a group including polyglycolic acid, polylactic acid, and polydiosanone. Absorbable materials are preferably used so that the flexible thread can be reabsorbed as the bone fuses. Alternatively, the flexible thread 60 may be selected from a group of non-absorbables such as nylon and polyproplene. As with newer sutures, the flexible thread 60 may also be coated with antimicrobial substances to reduce the chances of wound infection. Good suture type materials include commercial materials such as MONOCYRL™ (poligelcaprone), VICRYL™ (polyglactin), PDS™ (polydiodioxanone) made by ETHICON (Johnson & Johnson). For greater pulling strength, the flexible thread 60 may be made of stronger fibers such as aramid fabrics including DuPont's Kevlar® or Nomex® polyethylene fibers. For superior pulling strength, the flexible thread can also be metallic wire 60 made from metals similar to bone anchor assemblies, such as stainless steel or titanium.

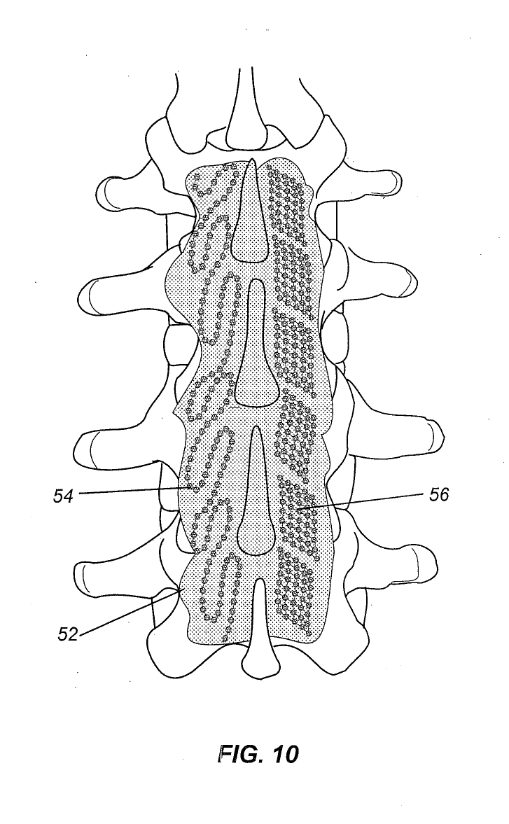

As shown in FIG. 10, the biocompatible string 54 can also be laid or looped throughout a posterolateral bone fusion bed 52 to help hold the fusion bed together until such time that the fusion is complete. Optionally, the bio-compatible mesh 56 may be laid onto a bone fusion bed 52 to form a matrix. This preferred mesh 56 embodiment may help the surgeon to support and hold the bone graft together while putting the rigid spinal bone screw and rod assembly together.

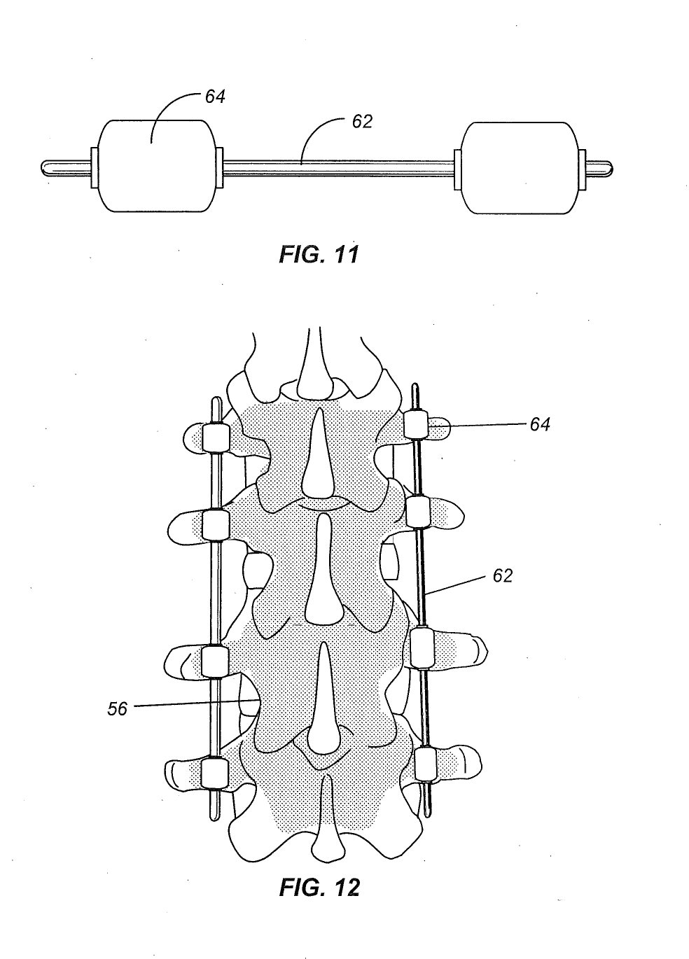

In another posterolateral fusion use, the embodiment shown in FIG. 11 may help stabilize the spine during the fusion process. In this preferred embodiment, a flexible rod 62 slides through bio-compatible material made from silicon nitride blocks 64. These blocks 64 are preferably cylinderal in shape with rounded edges. They may also be cubical. These blocks 64 slide along the flexible rod 62 so that they can be positioned on the spinous processes of the vertebrae. These blocks 64 are preferably larger than the ceramic spheres or shells and their size depends on the spinal vertebrae to be fused. The length of the blocks 64 for cervical vertebrae can be, for example, as small as 5 mm whereas the lumbar vertebrae blocks can be larger than 1 cm. The diameter of the blocks 64 also depends on the size of the spinous process. The flexible rod 62 is preferably made of polyether ether ketone (PEEK) or stainless steel cable. The length of the flexile rod 62 also depends on the number of vertebrae to be fused. The range can vary from 5 to 20 centimeters (cm). The blocks 64 are then interspersed along the flexible rod 62. The blocks 64 can be laid in between the decorticated spinous processes. During the fusion process, these blocks 64 fuse to the spinal processes and the flexible rod 62 to restrict the motion between vertebrae thereby stabilizing the spine during and after the fusion process. FIG. 12 shows how these flexible rods 62 can be laid in between the decorticated spinous processes while bone graft fusion bed 56 is being laid on top of the posterolateral vertebrae.

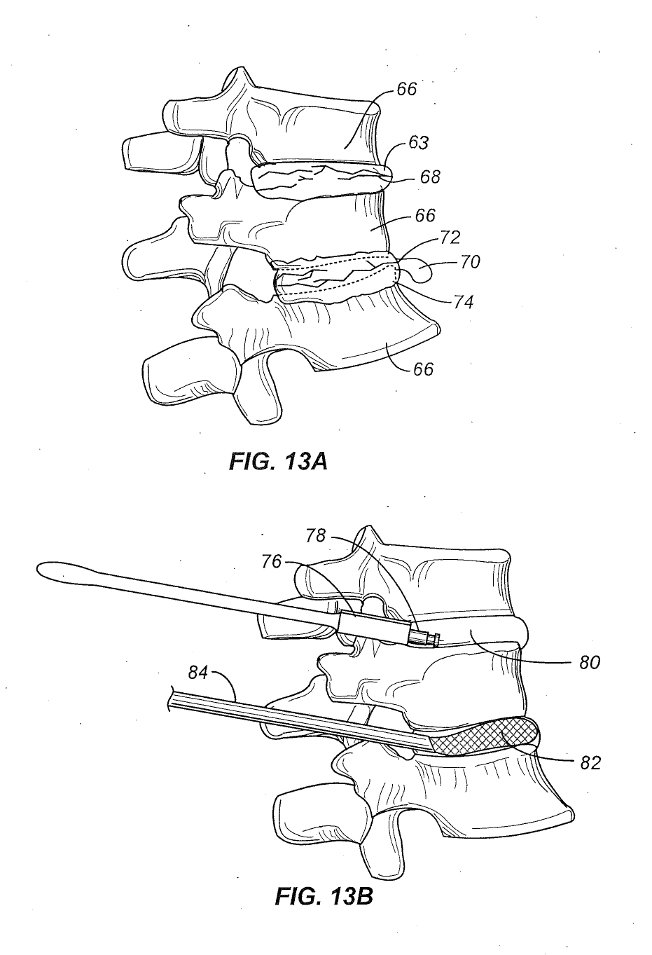

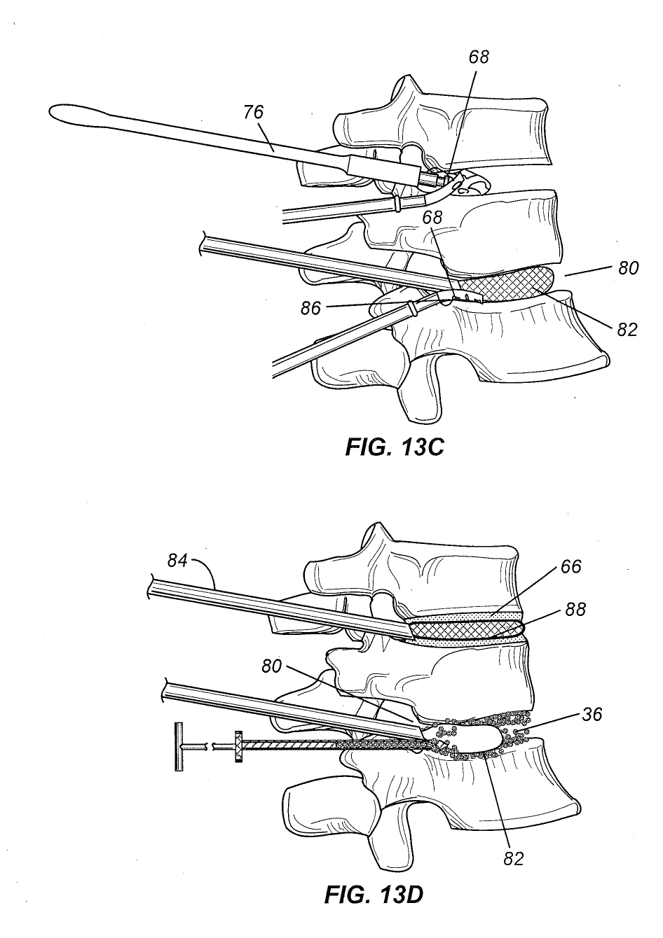

In another preferred embodiment, the present invention includes both the bio-compatible material and a device, as well as a related method, for repairing (e.g. replacing in whole or in part) an intervertebral disc by delivering the biocompatible material in situ from the posterior side of the spine. As mentioned above and shown in FIG. 13A, the intervertebral discs 63 are located between adjacent vertebrae 66 in the spine and provide structural support for the spine as well as the distribution of forces exerted on the spinal column. The intervertebral disc 63 contains stiffer outer portion (catheter fibrosus) that serves to provide peripheral mechanical support and torsional resistance. An inner portion (nucleus pulpous) contains a softer nuclear material to resist hydrostatic pressure. Most intervertebral discs, however, are susceptible to a number of injuries. With advancing age and constant pressure, disc herniation 68 is common. During herniation, spinal column pressure compresses the discs. Herniation starts when the nucleus 70 begins to extrude through an opening 72, often where the herniated disc 74 impinges on nerve roots in the spine. In most cases, the posterior and posterolateral portions of the discs are the most susceptible to such herniation.

Over time, herniated discs 74 begin to lose their height and the disc space between vertebrae is reduced. If surgical intervention is chosen, the surgeon first performs a bilateral hemilaminectomy, that is, removes the budging nucleus 70 and outer sections of herniated discs 74. To reach the inner disc nucleus between the vertebrae, a disc space distractor 76 shown in FIG. 13B is used. The current disc space distractors 76 used by surgeons today are similar to a metal chisel. A distractor tip 78 is slipped into the intervertebral disc space 80 and the distractor 76 is rotated to separate and increase the disc space between opposing vertebrae. This procedure opens the disc space while the distractor 76 maintains its position and height while the surgeon removes the herniated disc 74.

In a preferred embodiment, the present invention includes the methods of providing, inserting, and positioning an inflatable balloon 82 into the intervertebral disc space 80 from the posterior side of the spine. As shown in FIG. 13B, the balloon 82 is preferably provided in collapsed form and delivered into the intervertebral disc space 80 by the use of a catheter 84 that contains the balloon in a compact form within its proximal portion. In a preferred embodiment, the balloon 82 provides both an exterior tissue contracting surface and an interior cavity. The balloon 82 is preferably inflatable, expandable and deflatable by the delivery of gas under pressure and/or by the delivery of fluid materials. The balloon 82 may be introduced and positioned in various different areas of the disc space 80 by inflating and deflating fluid or gas pressure within the balloon 82 to achieve optimal intervertebral distraction. Whereas a metal distractor 76 maintains a static position, the balloon 82 can move dynamically throughout the intervertebral disc space 80 while maintaining a constant disc height between the vertebrae. As shown in FIG. 13C, the balloon 82 allows the surgeon unobstructed access to the intervertebral space 80 while he/she cuts and removes the rest of the disc herniation 68 with a rotate cutter 86. In contrast, a surgeon using a conventional distractor 76 must cut around the distractor 76 whereas the balloon 82 distractor allows the surgeon to cut above or under the balloon 82. The same ease-of-use of the balloon 82 distractor may also work well while decortifying the vertebrae endplates with scrapers and chisel. Additionally, the balloon 82 distractor may be more convenient when laying bone graft throughout the intervertebral disc space 80.

When filled, the balloon is preferably a semi-flattened ovoid shape, wherein the length and width of the balloon are greater than its height, and length greater than width. Whether the surgeon uses solid implants, solid implants with biocompatible material, biocompatible material alone or a balloon prosthesis while grafting, the balloon 82 can be filled with conventional materials and components available in the art. If the balloon 82 is used to place solid implants 88 or biocompatible material 36 is injected directly into the intervertebral disc space 80 shown in FIG. 13 D, a more compliant non-absorbable material such as polymeric materials can be used. If a balloon prosthesis is desired, the balloon 82 material and components can be made with more bioactive material, but less compliant absorbable material, known in the art to facilitate bone growth and differentiation for easier integration into the host tissue. In one embodiment, the balloon can be constructed from a more porous material where pores sizes can retain the biocompatible material 36 but permit the passage of graft 66 or other biomaterials promote fusion differentiation and growth. In another embodiment, the balloon 82 can be made of both an interior compliant expandable balloon to restore the intervertebral space and a more noncompliant porous exterior balloon to deliver bioactive material.

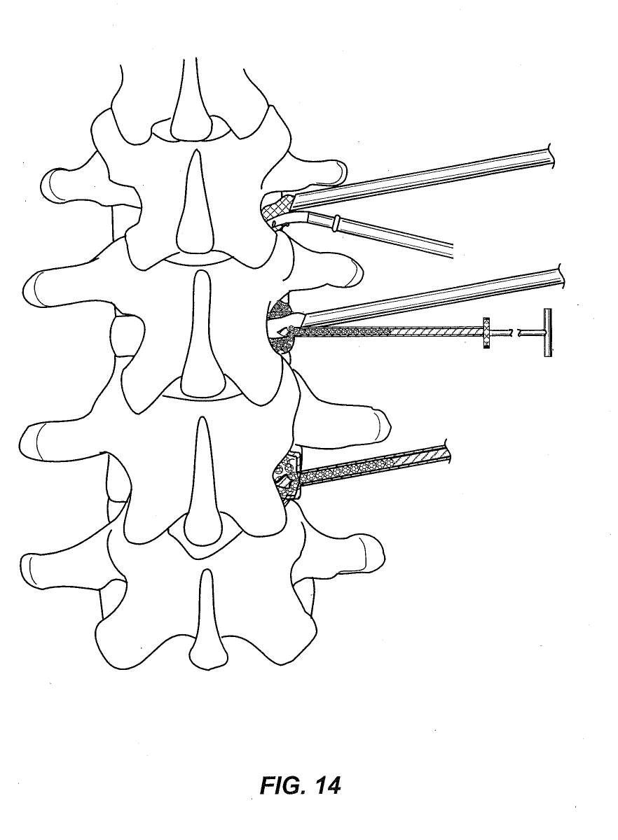

In another preferred embodiment, the bilateral hemilaminectomy and discectomy procedures and the placement of the biocompatiable material and balloon prosthesis can be performed on either lateral side of the spine as shown in FIG. 14. The right lateral side is shown. The above procedures and placement can also be performed from the more difficult anterior side of the spine.

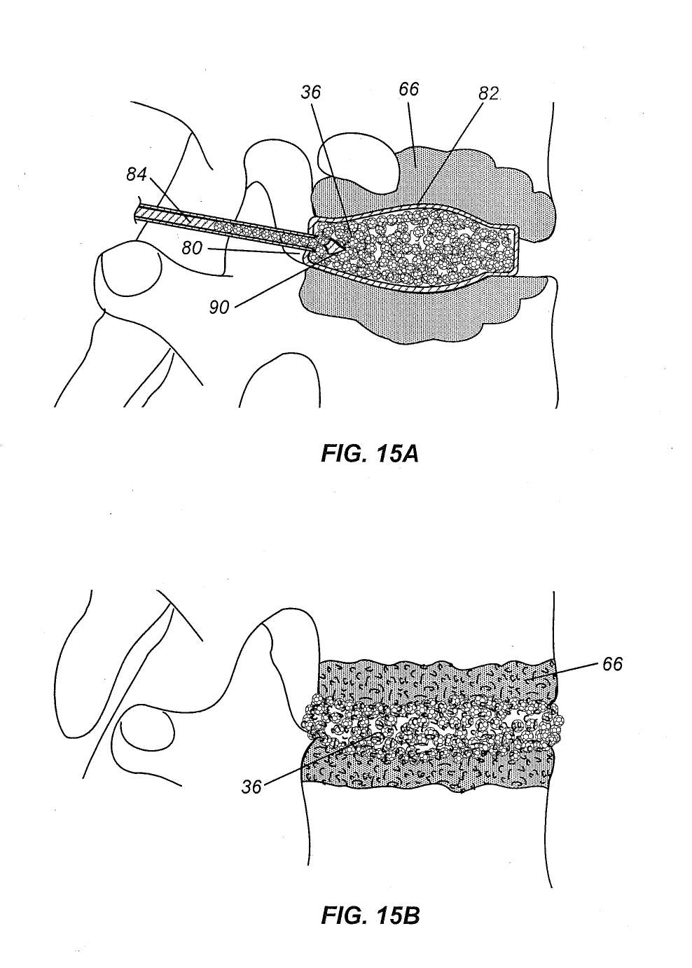

In an alternative embodiment shown in FIG. 15A, the balloon 82 can also be used to replace a total intervertebral disc by becoming a balloon prosthesis 90. Once positioned in the intervertebral disc space 80, the balloon 82 can be filled through a catheter 84 with bone graft 66 or biocompatible material 36 or, preferably, both. As illustrated in FIG. 15A, a balloon prosthesis 90 is created that replaces the intervertebral disc and/or solid intervertebral implants. A surgeon can then expand the balloon prosthesis 90 to the desired anatomy, height and function of the original disc. While the vertebrae are undergoing fusion, the balloon prosthesis 90 can maintain intervertebral distraction and, at the same time, promote bone growth and differentiation. The balloon prosthesis 90 may possess a similar or closer load bearing, compressive and mechanical properties to that of the original intervertebral disc, especially in comparison to implants made from materials such as metal, ceramics, cements or polymers. While the vertebrae are undergoing fusion, a balloon prosthesis 90 shown in FIG. 15B may be made from absorbable material and selected from a group including polyglycolic acid, polylactic acid, and polydiosanone. With such absorbable material, a mix of biocompatible material 36 and bone graft 66 is left behind to promote bone differentiation and growth during vertebrae fusion.

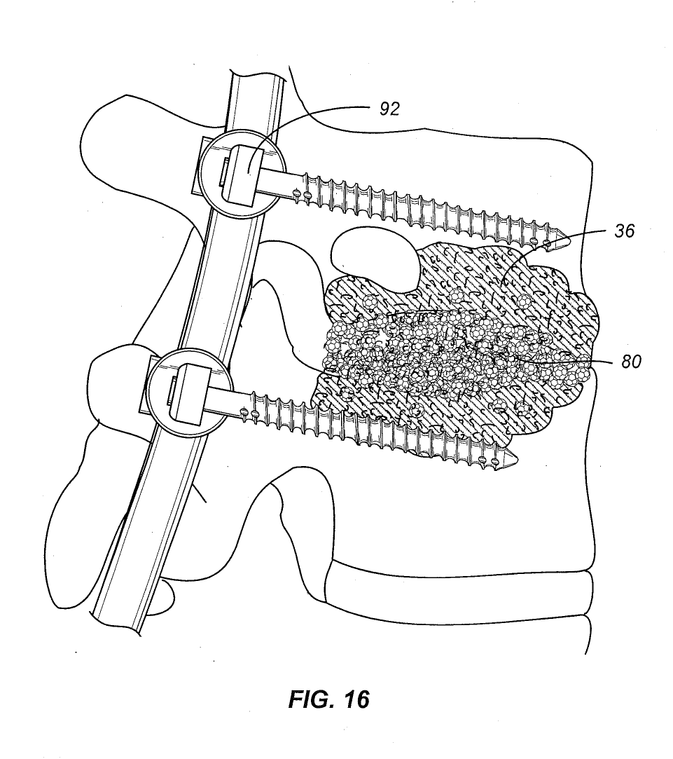

Whether the biocompatible material 36 is used in a balloon prosthesis or alone to fill the intervertebral disc space 80, the various embodiments of the present invention in FIG. 16 may be further supported and enhanced by a supplemental segmental internal fixation device 92, especially, during fusion.

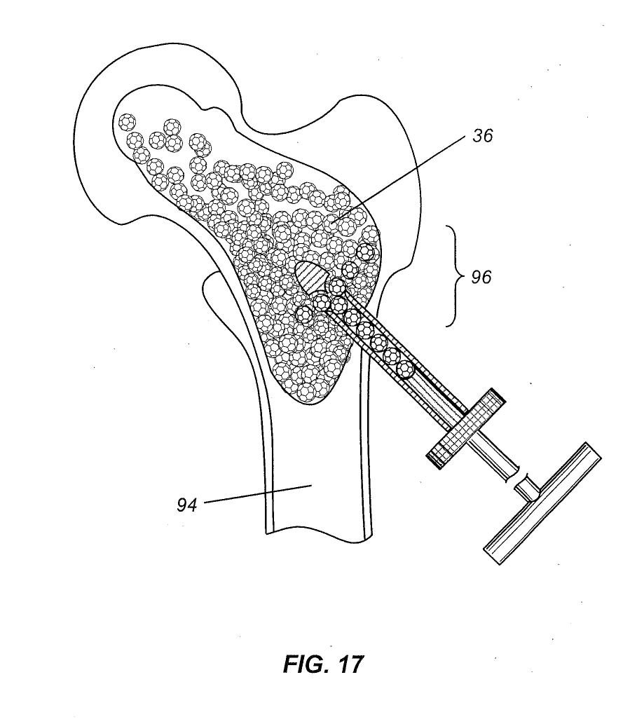

Now turning to FIG. 17, the bio-compatible material 36 can be also used for in vivo filing of defects, cavities or gaps in bones. A good example of this is the filling of a long bone such as the femur bone 94. Femoral neck fractures are frequent in osteoporotic bone and usually require hip screws or plates. In many cases, these procedures reduce the mechanical strength of the femoral neck 96. It has been reported that filling the proximal femur with bone cement effectively strengthens the femoral neck with limited risk of shear stresses. As described earlier, the use of prior art bone cements, such as PMMA or PU, is unpredictable for long-term use. The use of the bio-compatible material 36 of the present invention may not only strengthen a femoral neck fracture but may also augment the bone through increased bone differentiation and growth to prevent additional fractures. Furthermore, the percutaneous injection of the bio-compatible material 36 into a weakened femoral neck 96 before a fracture occurs may prophylactically strengthen an intact osteoporotic femoral neck that is a high risk of fracture.

E. Instruments

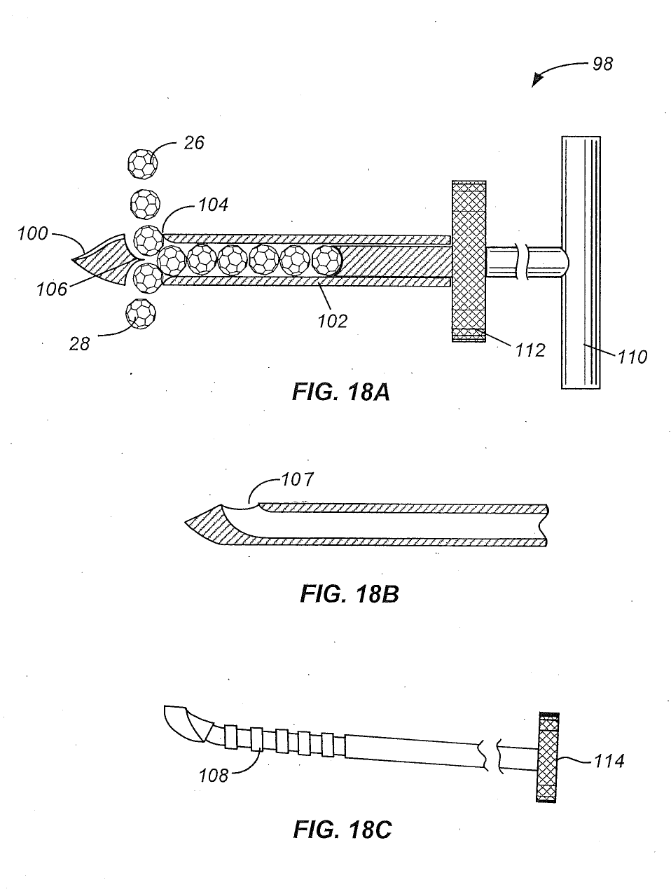

Since cancellous bone resists the injection of substances like bone cement, small diameter needles or catheters are typically used and extremely high pressures are required to force the bone cement through the needles or catheters into vertebral bodies. To solve this problem, a new embodiment of the present invention is shown in FIG. 18A. It is a surgical instrument where ceramic spheres 26 or ceramic shells 28 can be loaded and injected into a bone defect, cavity or gap. Since boring is the process of enlarging a hole that has already been drilled, the sphere gun 98 consists of a tool bit 100 at its proximal end to cut into the bone. After the tool bit 100, the sphere gun 98 may have a larger diameter catheter 102 into which the ceramic spheres 26 or shells 28 are loaded. In a preferred embodiment, the catheter may be double-sided 104 with two opposing exit holes. A taper point 106 separates the ceramic spheres 26 or shells 28 to either side as they exit the catheter 102. Doubled-side 104 catheter holes may be used when the cavity is large and where it is best to apply pressure equally. This may be particularly important during vertebroplasty when a surgeon is trying to restore the pre-fracture anatomy of a crushed or fractured vertebra to both the anterior and posterior sides or the superior (top) or inferior (bottom) vertebral plates.

In another preferred embodiment, a single-sided exit hole 107 shown in FIG. 18B may work better when a surgeon wants to, for example, push the bio-compatible material up into the neck of a femur (FIG. 17). In other embodiment, a flexible catheter 108 shown in FIG. 18C may help reach and position the biocompatible material into hard-to-reach places.

In all of the preferred sphere gun 98 embodiments, the distal end comprises a plunger 110 and finger hold 112 to exert pressure and push the ceramic spheres 26 or shells 28 out the double-sided 104 or one-sided 107 exit holes (FIG. 18A, B). On the steerable sphere gun (FIG. 18C), a steering knob 114 may be added before or after the plunger 110 knob. A steerable sphere gun may be especially useful while working in intervertebral disc spaces. In another embodiment, a pressure gauge (not shown) may be added to help avoid extreme resistance or pressure avoiding further damage or fracture. For convenience sake, a cartridge of ceramic spheres 26 or shells 28 containing the appropriate sized ceramic spheres, ceramic shells, bio-materials and fillers can be pre-manufactured and loaded into the sphere gun 98 when needed.

E. Kits

The first, second and third components are preferably combined to make a kit that contains the biocompatible composite. A sterile syringe or injection device is preferably included in such a kit. The injection device may, for example, include the aforementioned sphere gun. Such a kit avoids the measurement, mixing, filling and choosing the appropriate device for application, which is a major convenience to the surgeon.

F. Advantages

The preferred biocompatible material and, in particular, the silicon nitride ceramic of the present invention provides at least the following advantages over the prior art and, especially over the use of PMMA or PU:
a biocompatible material that is a permanent replacement for osteoporotic, compressed, fractured bones or intervertebral discs;
a biocompatible material that is bioinert, osteoconductive, osteoinductive and promotes bone differentiation and growth;
a biocompatible material that can be fabricated into various shapes and tailored to many orthopedic uses;
a biocompatible material that does not fracture under normal physiologic loading;
a biocompatible material that does not exude toxic plasticizers; and
a biocompatible material that is radioopaque.

A variety of further modifications and improvements to the biocompatible materials of the present invention will be apparent to those persons skilled in the art. In this regard, it will be recognized and understood that the biocompatible material can be changed to different compositions, shapes and sizes along with different biomaterials to augment bone growth and differentiation. Accordingly, no limitations on the invention is intended by way of the foregoing description and accompanying drawings, except as set forth in the appended claims.

What is claimed is:

1. A biocompatible material for orthopedic uses comprising porous ceramic spheres, including polyhedrals, having compressive strength corresponding substantially with natural cancellous bone.

2. The biocompatible material of claim 1 wherein said porous ceramic spheres are formed from doped silicon nitride.

3. The biocompatible material of claim 2 wherein the doping for said silicon nitride is selected from the group consisting of yttrium oxide, magnesium oxide, strontium oxide, alumina, and/or combinations thereof.

4. The biocompatible material of claim 1 wherein said porous ceramic sphere has a porosity of 10% to 50% by volume and pore sizes ranging from 5 to 500 microns.

5. The biocompatible material of claim 1 wherein said porous ceramic spheres are polyhedral in shape.

6. The biocompatible material of claim 5 wherein said polyhedral is selected from the group consisting of hexagons, octahededrons, and combinations thereof.

7. The biocompatible material of claim 1 wherein the diameters of said ceramic spheres is in a range of about 1 millimeter to about 5 millimeters.

8. The biocompatible material of claim 1 wherein said compressive strength ranges between 10 MPa and 40 MPa.

9. The biocompatible material of claim 1 wherein said porous ceramic spheres are formed from silicon nitride, its analogs or derivatives.

10. The biocompatible material of claim 1 wherein said porous ceramic spheres are coated with biomaterials selected from the group consisting of calcium phosphate, hyroxyapatite, tri-calcium phosphate and/or de-mineralize bone.

11. The biocompatible material of claim 1 wherein said porous ceramic spheres are impregnated with osteoconductive and/or osteogenic bio-materials selected from the group consisting of bone morphorgenic proteins, growth factors, bone marrow aspirate, stem cells, progenitor cells, amniotic fluid and/or antibiotics.

12. The biocompatible material of claim 1 further comprising a liquid or gel substance to hold said bio-compatible material together.

13. The biocompatible material of claim 12 wherein said liquid or gel is selected from the group consisting of collagen, glycoaminoglycans, hyrodgels and/or combinations thereof.

14. The biocompatible material of claim 1 wherein said ceramic spheres are threaded together.

15. The biocompatible of claim 14 wherein said ceramic spheres are threaded together in a mesh.

16. The biocompatible material of claim 1 wherein flexible rods are placed through ceramic blocks of said bio-compatible material.

17. The biocompatible material of claim 16 wherein said flexible rods are comprised of PEEK or stainless steel cable.

18. The biocompatible material of claim 1 wherein said biocompatible material is placed into intervertebral disc space.

19. A method for delivering biocompatible material into a vertebral body comprising:
selecting a balloon adapted to be delivered to an intervertebral disc space through a catheter;
said balloon having an interior cavity expandable with gas or fluid; and
filling said balloon with a bioactive biomaterial and delivering said bioactive material into a vertebral body.

20. The method of claim 19 wherein said balloon is an intervertebral disc distractor.

21. The method of claim 19 wherein said balloon is an intervertebral disc prosthesis in situ.

22. The method of claim 19 wherein said intervertebral disc balloon prosthesis is a partial or total intervertebral disc prosthesis.

23. The method of claim 19 wherein said balloon is porous.

24. The method of claim 19 wherein said balloon is formed from a biogradable material.

25. The method of claim 19 wherein said bioactive biomaterial is silicon nitride.

26. The method of claim 19 wherein said bioactive biomaterial permits the balloon to be inflated to the entire volume of an intervertebral disc space.

27. A medical instrument comprising a catheter and a plunger that injects biocompatible materials into bone defects, cavities or gaps.

28. The medical instrument of claim 27 wherein said catheter has a one-sided exit hole.

29. The medical instrument of claim 27 wherein said catheter has double-sided exit holes.

30. The medical instrument of claim 27 wherein said catheter has a flexible catheter tube.

31. The medical instrument of claim 30 further comprising a steering knob.

32. The medical instrument of claim 27 further comprising biocompatible polyhedrals that can be injected into body orifices.

33. A method for augmenting and restoring osteoporotic, compressed or fractured vertebra comprising the steps of:
creating an entry hole to access a damaged portion of a vertebrae;
inserting a balloon catheter through said entry hole;
inflating said balloon;
deflating said balloon to form cavity; and
inserting a biocompatible material in the form of ceramic spheres through said catheter and into said cavity.

34. A method for fusing osteoporotic, compressed or fractured vertebrae comprising the steps of inserting ceramic spheres or ceramic shells formed of bio-compatible material into defects, cavities, gaps or fusion beds in a human body.

35. The method for fusing osteoporotic, compressed or fractured vertebrae of claim 33 wherein said ceramic spheres or ceramic shells are connected by one or more strings.

36. The method for fusing osteoporotic, compressed or fractured vertebrae of claim 33 wherein said ceramic spheres or ceramic shells and their connecting strings form a mesh.

37. The method for fusing osteoporotic, compressed or fractured vertebrae of claim 33 wherein flexible rods connect said ceramic spheres or ceramic shells.

38. A method for filling a cavity in a bone comprising the steps of:
creating an entry hole to access a cavity in said bone;
inserting a catheter through said entry hole;
injecting a biocompatible material in the form of ceramic spheres through said catheter and into said cavity.

39. A method for restoring, repairing and fusing intervertebral discs comprising the steps of:
inserting a balloon into intervertebral disc space;
inflating said balloon;
restoring intervertebral disc space;
delivering bioactive biomaterial into the intervertebral disc space using said balloon;
supporting and enhancing intervertebral disc space with a supplemental segmental internal fixation device.
